(19) United States
(12) Patent Application Publication
(10) Pub. No.: US 2017/0215800 A1
(43) Pub. Date: Aug. 3, 2017

(54) WEARABLE DEVICE

(71) Applicant: ZENTAN TECHNOLOGY CO., LTD., NEW TAIPEI CITY (TW)

(72) Inventor: TONG-PIE CHEN, NEW TAIPEI CITY (TW)

(21) Appl. No.: 15/178,312

(22) Filed: Jun. 9, 2016

(30) Foreign Application Priority Data
Jan. 29, 2016 (TW) 105201421

(51) Int. Cl. A61B 5/00; A61B 5/0408

(52) U.S. Cl. CPC A61B 5/6831; A61B 5/04085; A61B 2560/0468; A61B 2562/0209; A61B 2562/125; A61B 2562/164

(57) ABSTRACT

Disclosed is a wearable device which includes a tape-like fabric body, at least one electrically conductive unit, at least one electric signal detection unit, an anti-slip structure, and a data transmission unit. The electrically conductive unit includes at least one conductive member arranged to pass through a first surface and a second surface of the tape-like fabric body. The electric signal detection unit and the data transmission unit are arranged on the first and second surfaces respectively. The electric signal detection unit includes a sensing portion configured to contact a body surface of a living body to detect micro electric signals and a connection portion configured to be electrically connected to the conductive member and transmit the micro electric signals to a terminal device via the data transmission unit. The anti-slip structure is configured to come in contact with the body surface together with the sensing portion.

### BACKGROUND OF THE INVENTION

1. Field of the Invention

The instant disclosure relates to a physiological signal detection belt; in particular, to a wearable device for detecting physiological signals which cannot break away easily from its attachment to the body surface of a living body.

2. Description of Related Art

The conventional detection belt for detecting physiological signals that can be worn on a living body (e.g., user's hand) by binding bands or fasteners is well known. The conventional detection belt can always detect physiological signals (e.g., heartbeat) of the living body by using its electrode assembly to contact the body surface (e.g., user's skin). The conventional detection belt not only is used by special patients, but also is widely used by various athletes to monitor their physiological signals.

However, there are many problems with using the conventional detection belt to detect physiological signals. One of the most serious problems is that the sensing portion of the conventional detection belt may easily break away from its attachment to the body surface (e.g., user's skin) during a long-term motion or a strenuous motion, and this will result in deficient detection. Hence, there is an urgent need for technical features to solve the deficient detection caused by a breakaway separation.

### SUMMARY OF THE INVENTION

The object of the instant disclosure is to provide a wearable device for detecting physiological signals to solve the deficient detection caused by a breakaway separation during a long-term motion or a severe motion.

The benefits of the present invention include: because the anti-slip structure is configured to come in contact with the body surface of a living body together with the sensing portion of the corresponding electric signal detection unit, the wearable device cannot break away easily from its attachment to the body surface upon the movement of the living body.

### BRIEF DESCRIPTION OF THE DRAWINGS

FIG. 1 is an exploded diagram of a wearable device for detecting physiological signals according to a preferred embodiment of the present invention;

FIGS. 2-4 are partially assembled diagrams of the wearable device according to a preferred embodiment of the present invention;

FIG. 5 is an assembled diagram of the wearable device according to a preferred embodiment of the present invention;

FIG. 6 is a front-side schematic diagram of the wearable device according to a preferred embodiment of the present invention;

FIG. 7 is a cross-sectional diagram of an electric signal detection unit of the wearable device according to a preferred embodiment of the present invention;

FIG. 8 is an exploded diagram of a wearable device for detecting physiological signals according to another preferred embodiment of the present invention;

FIGS. 9 and 10 are schematic diagrams showing different aspects of an anti-slip structure of the wearable device according to another preferred embodiment of the present invention;

FIG. 11 is an exploded diagram of a wearable device for detecting physiological signals according to still another preferred embodiment of the present invention; and

FIG. 12 is a front-side schematic diagram of the wearable device according to still another preferred embodiment of the present invention.

### DETAILED DESCRIPTION OF THE PREFERRED EMBODIMENTS

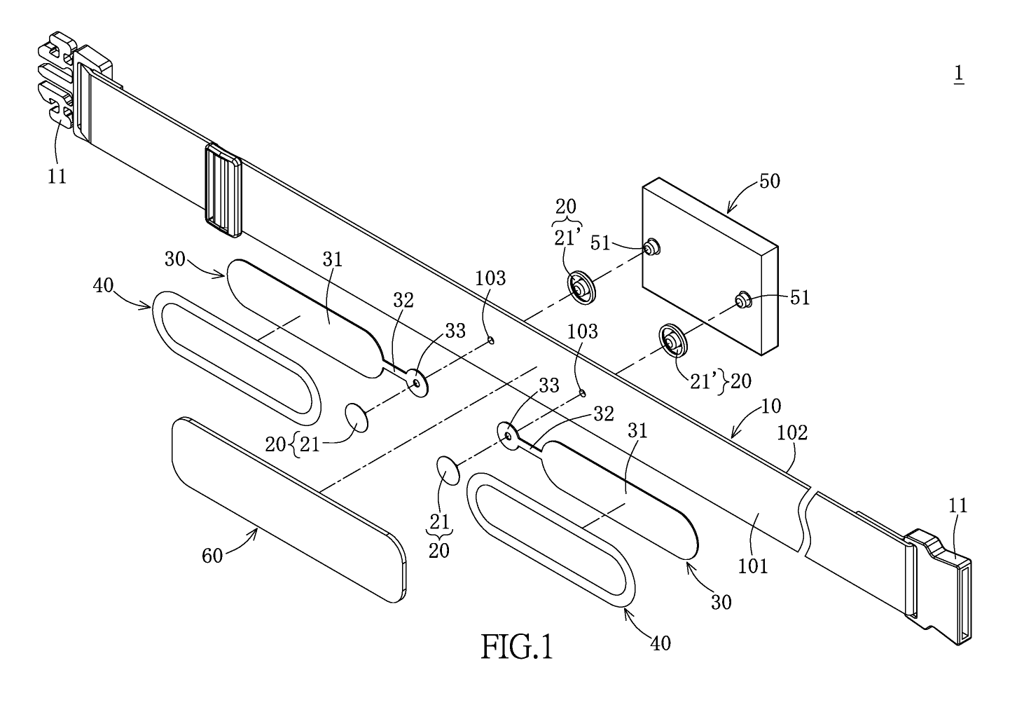

Please refer to FIGS. 1 to 5. The wearable device 1 includes a tape-like fabric body 10, two electrically conductive units 20, two electric signal detection units 30, two anti-slip structures 40, and a data transmission unit 50. The electric signal detection units 30 and the anti-slip structures 40 are arranged on one side of the tape-like fabric body 10, and the data transmission unit 50 is arranged on the other side of the tape-like fabric body 10. The electrically conductive units 20 are arranged to pass through the tape-like fabric body 10 such that the electric signal detection units 30 can be electrically connected to the data transmission unit 50. The electric signal detection units 30 is configured to come in contact with a body surface of a living body to detect micro electric signals, and then transmit the micro electric signals to the data transmission unit 50 via the electrically conductive units 20. The data transmission unit 50 is configured to convert the micro electric signals into output digital signals, and then transmit the output digital signals to a terminal device such as a computer or smart phone via wire or wireless communications. Please note, in the instant embodiment, the wearable device 1 includes two electric signal detection units 30 and two anti-slip structures 40, however they are not restricted thereto. For example, the wearable device 1 may include only one electric signal detection unit 30 and only one anti-slip structure 40.

Specifically, the tape-like fabric body 10 has a first surface 101 and a second surface 102 opposite to the first surface 101. The two ends of the tape-like fabric body 10 each have a connection member 11 arranged thereon, and the tape-like fabric body 10 can be shaped from a linear shape into the ring-like shape by fastening the two connection members 11 together. In practice, the tape-like fabric body 10 can be worn on a living body (e.g., human body) in its ring-like shape. In the instant embodiment, the tape-like fabric body 10 can include a luminous fiber, an antibacterial fiber, a thermochromic fiber, etc., according to the required basic or end product. There is no particular restriction on the material which may be used for the tape-like fabric body 10. Each connection member 11 can be any type of conventional fastener such as, but not limited to, Velcro fastener and magnetic fastener.

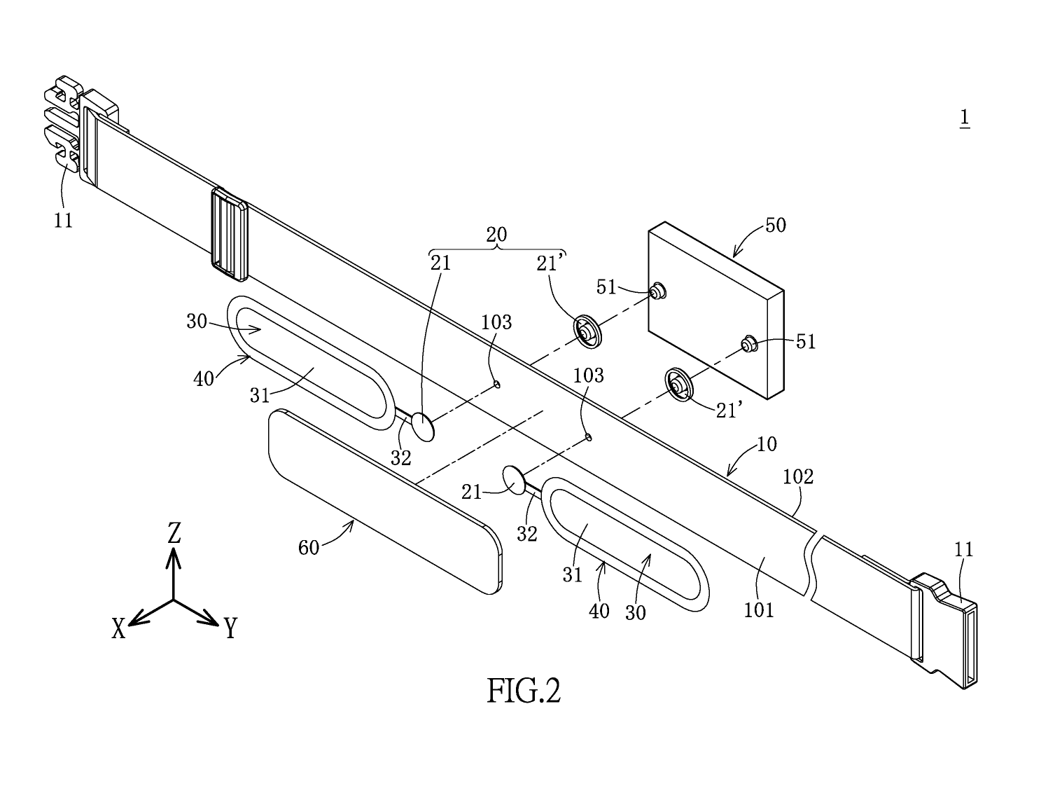

Each electrically conductive unit 20 is arranged through the tape-like fabric body 10, and includes a plurality of conductive members 21, 21′. The conductive members 21, 21′ are configured to be installed on the first and second surfaces 101, 102 of the tape-like fabric body 10 respectively and electrically connected to each other. The tape-like fabric body 10 is thus formed with a plurality of through holes 103 such that the electrically conductive unit 20 can be arranged to pass through the tape-like fabric body 10. In the instant embodiment, all of the conductive members 21, 21′ are metal conductive structures, wherein each conductive member 21′ on the second surface 102 is used in relation to a corresponding electrical connection structure 51 of the data transmission unit 50. For example, each conductive member 21′ and the corresponding electrical connection structure 51, as shown FIGS. 1 and 2, are configured to be fastened to each other. Similarly, each conductive member 21 on the first surface 101 is used in relation to a corresponding connection portion 33 of the electric signal detection unit 30, as described below. Please note, each electrically conductive unit 20 may include only one conductive member (e.g., conductive member 21′ shown in FIGS. 1 and 2) installed on the second surface 102. The conductive member 21′ is arranged to pass through the tape-like fabric body 10 to be fastened to the connection portion 33, there is no particular restriction on the structure of the conductive member 21′.

Each of the two electric signal detection units 30 is made from an electrically conductive fabric, and is arranged on the first surface 101 of the tape-like fabric body 10 at a predetermined interval. In addition, the contour and the fiber material of each electric signal detection unit 30 can be changed according to the required basic or end product. For example, each electric signal detection unit 30 can further include any functional fiber such as, but not limited to, luminous fiber, antibacterial fiber, and thermochromics fiber.

Each electric signal detection unit 30 has a sensing portion 31, an extension portion 32, and a connection portion 33. The extension portion 32 is connected to the sensing portion 31, and the connection portion 33 is connected to the extension portion 32. The contour of the sensing portion 31 is configured in relation to that of the tape-like fabric body 10, and the width along the z-axis as shown in FIG. 2 of the sensing portion 31 is smaller than that of the tape-like fabric body 10. The sensing portion 31 has an external surface (unnumbered) away from the first surface 101 which is used as a sensing surface for attaching to a body surface of a living body to detect its micro electric signals. The width of the connection portion 32 is smaller than that of the sensing portion 31. The connection portion 33 is electrically connected to the corresponding conductive member 21 such that micro electric signals detected by the sensing portion 31 can be transmitted to the data transmission unit 50 by the conductive members 21, 21′.

Referring to FIG. 2, the contour of each anti-slip structure 40 is designed in relation to the corresponding electric signal detection unit 30. For example, the two anti-slip structures 40 are circular in shape. In addition, the dimension of each anti-slip structure 40 is greater than that of the corresponding electric signal detection unit 30. In other words, the width along the z-axis as shown in FIG. 2 of each anti-slip structure 40 is greater than that of the corresponding electric signal detection unit 30.

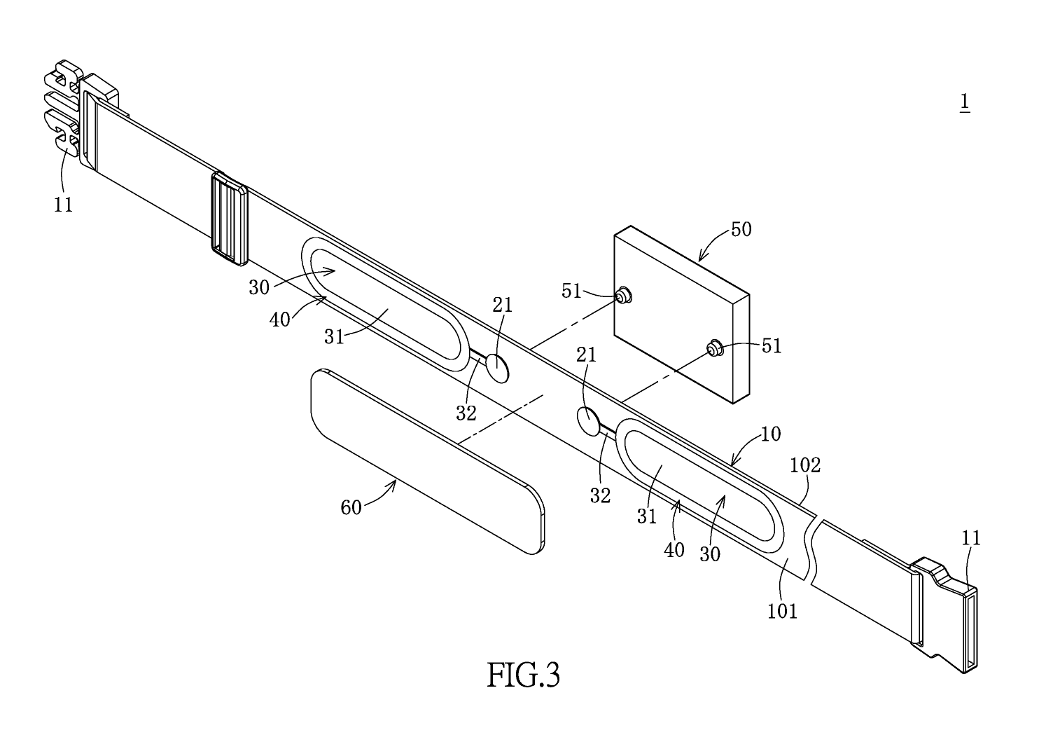

Referring to FIG. 3, each anti-slip structure 40 is arranged on the first surface 101 of the tape-like fabric body 10 and surrounds the corresponding electric signal detection unit 30 as a shield on the periphery of the corresponding electric signal detection unit 30 and as a part of the first surface 101. Thereby, the structural strength of each electric signal detection unit 30 can be increased. In the instant embodiment, because each electric signal detection unit 30 is made from an electrically conductive fiber, the corresponding anti-slip structures 40 can be used as an edge-closing structure thereof. In practice, each anti-slip structure 40 is configured to come in contact with the body surface of a living body together with the sensing portion 31 of the corresponding electric signal detection unit 30. Thereby, the sensing portion 31 of each electric signal detection unit 30 cannot break away easily from its attachment to the body surface due to movement of the living body. Preferably, each anti-slip structure 40 has a microstructure arranged on its external surface which is on the same side as the sensing surface of the sensing portion 31, such that the frictional force between the sensing portion 31 and the body surface can be increased.

Please note, each anti-slip structure 40 can be fixed on the periphery of the corresponding sensing portion 31, and then be installed on the first surface 101 of the tape-like fabric body 10 together with the electric signal detection unit 30. Moreover, after fixing each electric signal detection unit 30 on the first surface 101 of the tape-like fabric body 10, each anti-slip structure 40 can be installed on the first surface 101 and surrounds the corresponding electric signal detection unit 30. In addition, each anti-slip structure 40 and the corresponding electric signal detection unit 30 can be concurrently installed on the tape-like fabric body 10.

Based on the above description, the method and the materials for use in the manufacture of the anti-slip structure 40 can be changed according to users' requirements. For example, the anti-slip structure 40 and the electric signal detection unit 30 can be joined together and then installed on the tape-like fabric body 10, or the anti-slip structure 40 can be directly installed on the tape-like fabric body 10 with the electric signal detection unit 30 thereon. Moreover, the anti-slip structure 40 can be made from a fabric material and disposed on the first surface 101 of the tape-like fabric body 10 by adhering, ultrasonic welding, or sewing. In addition, the anti-slip structure 40 can be made from a coating material and coated on the first surface 101 of the tape-like fabric body 10.

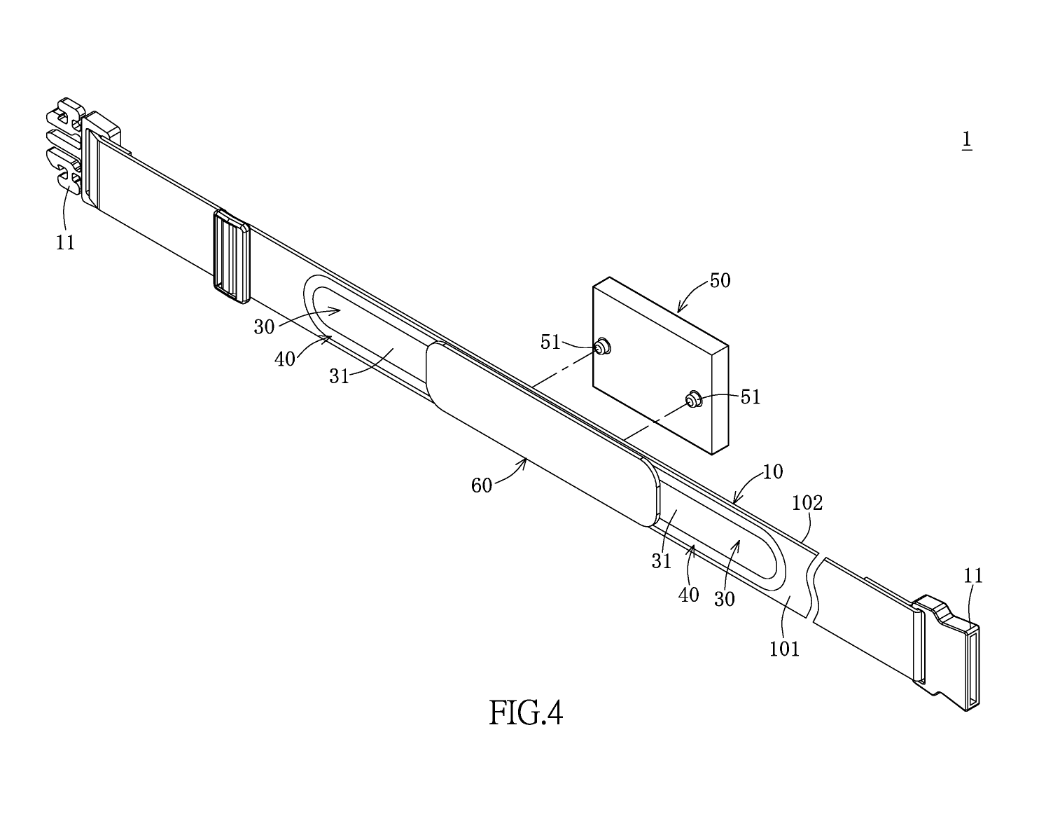

Referring to FIGS. 3 and 4, after installing the two electric signal detection units 30 and the two anti-slip structures 40 on the first surface 101 of the tape-like fabric body 10, the insulating member 60 is arranged on the first surface 101 to cover the connection portion 33 of each electric signal detection unit 30. Thus, the conductive members 21 on the first surface 101 cannot directly contact with the body surface of a living body. In the instant embodiment, the area of the insulating member 60 is greater than total area of the extension portion 32 and the connection portion 33 of each electric signal detection unit 30, and smaller than the area of the tape-like fabric body 10. Preferably, the area of the insulating member 60 is approximately equal to the area of each electric signal detection unit 30.

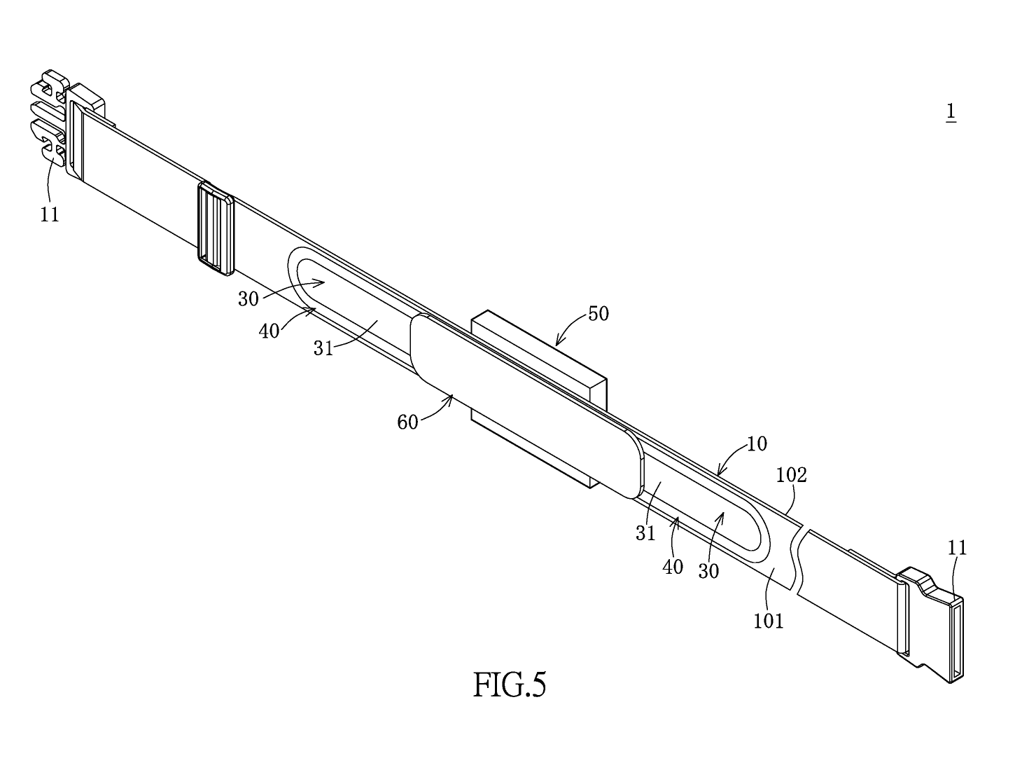

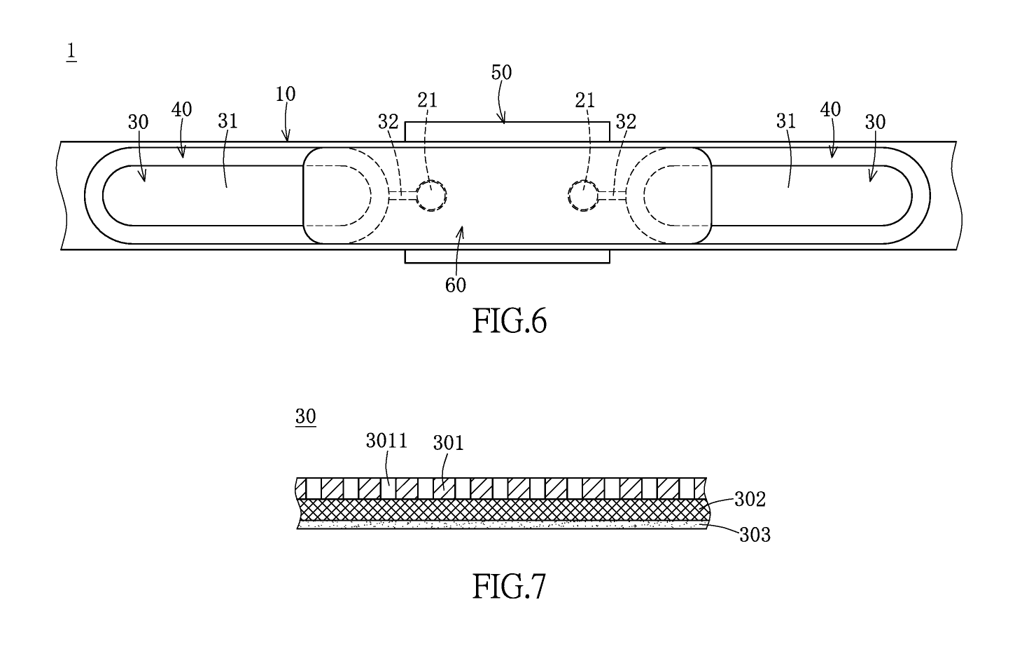

Referring to FIGS. 5 and 6, in practice, the width along the aforementioned transverse direction of each electric signal detection unit 30 is smaller than that of the tape-like fabric body 10. Each anti-slip structure 40 is disposed close to the boundaries of the tape-like fabric body 10. This will result in a relatively high detection efficiency of the wearable device 1.

Please refer to FIG. 7, which is a cross-sectional diagram of each electric signal detection unit 30. As shown in FIG. 7, each electric signal detection unit 30 is a composite material including a conductive layer 301, an interface layer 302, and a base layer 303. The conductive layer 301 and the base layer 303 are arranged on two opposite surfaces of the interface layer 302 respectively. In the instant embodiment, the conductive layer 301, the interface layer 302, and the base layer 303 can be made from the same or a different fabric, and laminated together by adhering, ultrasonic welding, or sewing, etc., wherein the conductive layer 301 is used to sense and transmit the micro electric signals.

Specifically, the conductive layer 301 can be made from an electrically conductive fabric, the interface layer 302 can be made from a water-absorbing fabric, and the base layer 303 can be made from an insulating fabric. An external surface of the conductive layer 301 away from the interface layer 302 is used as the aforementioned sensing surface, and an external surface of the base layer 303 away from the interface layer 302 is connected to the first surface 101. In practice, each electric signal detection unit 30 is flexible. The conductive layer 301 can be made using any suitable method, for example, it can be made from a blended fabric including fibers and metal fines, an electrospun fabric including metal nanoparticles, a blended fabric including a conductive polymer, or a base fabric coated with a conductive material.

Preferably, the conductive layer 301, as shown in FIG. 7, is formed with a plurality of micro-holes 3011. When the external surface (i.e., sensing surface) of the conductive layer 301 comes in contact with the body surface of a living body, sweat discharged from the body surface can pass through the micro-holes 3011 to be absorbed by the interface layer 302 and thus promote the detection efficiency of the conductive layer 301 upon detecting micro electric signals of the living body. In practice, the base layer 303 is capable of absorbing moisture.

The data transmission unit 50 is detachably installed on the second surface 102 of the tape-like fabric body 10, having two electrical connection structures 51 configured to be fastened to the two conductive members 21′ respectively. Thereby, the data transmission unit 50 can be electrically connected to the two conductive members 21′ by the two electrical connection structures 51, and receive the micro electric signals which are detected by the two electric signal detection units 30 from the two conductive members 21 on the first surface 101.

In other words, the micro electric signals detected by the sensing portion 31 of each electric signal detection unit 30 can be transmitted to the data transmission unit 50 by the electrically conductive units 20 (i.e., conductive members 21, 21′ on the first and second surfaces 101, 102), and converted into output digital signals by a corresponding converting module within the data transmission unit 50. After that, the output digital signals can be transmitted to a terminal device such as a computer and smart phone. Moreover, the data transmission unit 50 can include functional modules to assist conversion and transmission of the micro electric signals according to the required basic or end product. Specific examples of the function modules include processing module, power supply management module, signal converting module, data transmission module (e.g., Bluetooth/infrared data transmission module), filtering module, and signal amplifier.

Please note, although the data transmission unit 50 is detachably installed on the second surface 102 of the tape-like fabric body 10 by fastening the two electrical connection structures 51 to the two conductive members 21′ respectively, there is no particular restriction on the mechanism for installing the data transmission unit 50. For example, the data transmission unit 50 can be directly fixed on the second surface 102, such that the wearable device 1 can be used as a one-time product. Moreover, the data transmission unit 50 can be used with a replaceable battery, and it is thus reusable.

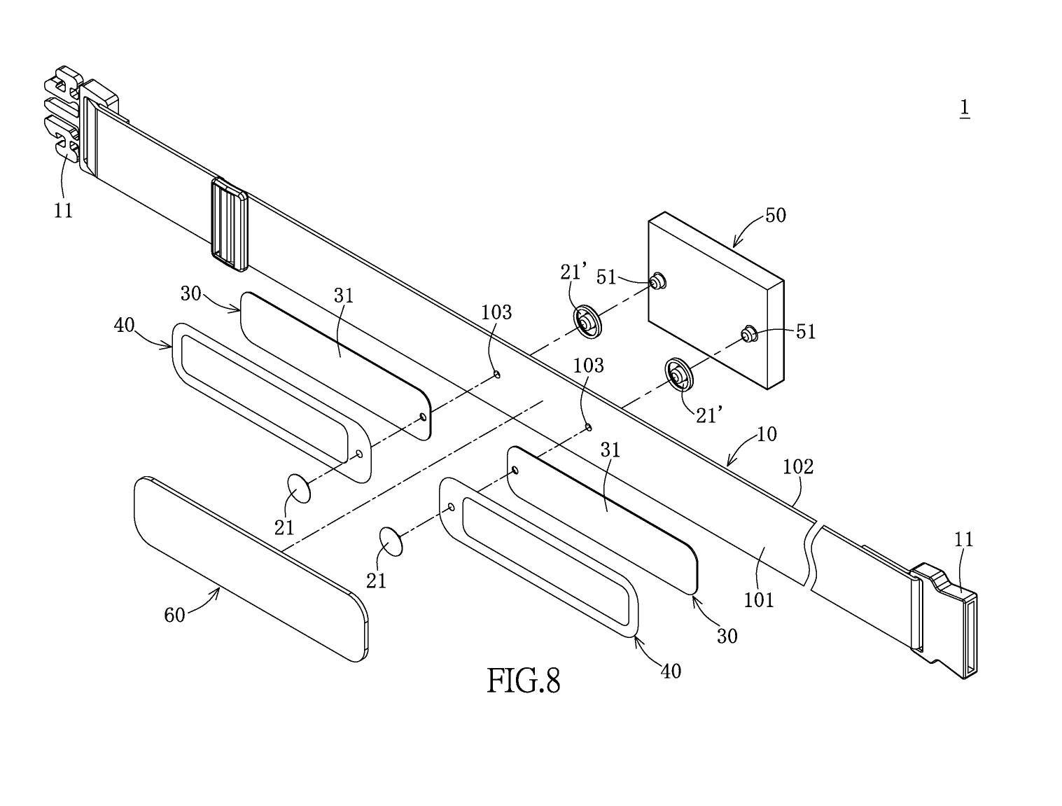

Please refer to FIGS. 8 to 10, each electric signal detection unit 30 can have various shapes, other units of the wearable device 1 are the same in structure and so will not be described here any further. As shown in FIG. 8, each electric signal detection unit 30 does not have the aforementioned extension portion 32 as shown in FIG. 2. Each of the two electric signal detection units 30 has the aforementioned connection portion 33, and the two connection portions 33 are positioned close to each other symmetrically. Each electric signal detection unit 30 is approximately rectangular in shape, having two approximately straight sides. However, each electric signal detection unit 30 can also be circular, oval, or square in shape, there is no particular limitation as to the shapes thereof.

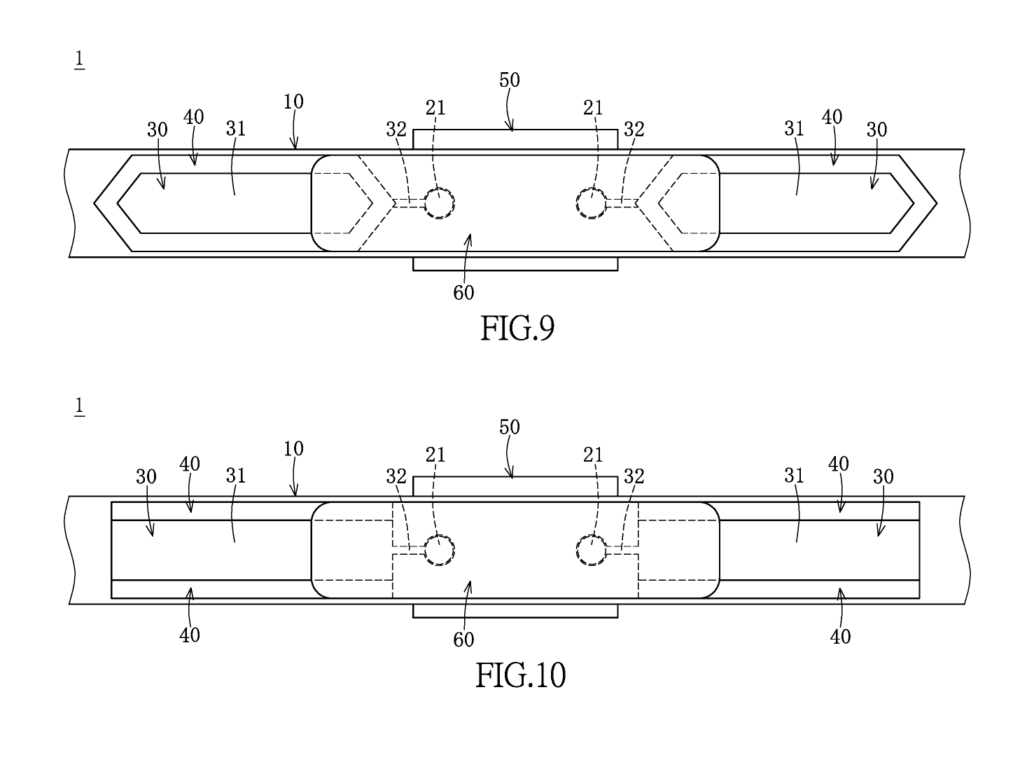

Referring to FIG. 9, the contour of the sensing portion 31 exposed from the first surface 101 of each electric signal detection unit 30 can be changed according to the required basic or end product, is not limited to those above mentioned. Each anti-slip structure 40 can conformally surround an outer periphery of the corresponding electric signal detection unit 30.

Referring to FIG. 10, each anti-slip structure 40 does not need to perfectly surround the outer periphery of the corresponding electric signal detection unit 30. In other words, each anti-slip structure 40 only surrounds a part of the outer periphery of the corresponding electric signal detection unit 30, and there is no particular restriction on the position of attaching each anti-slip structure 40.

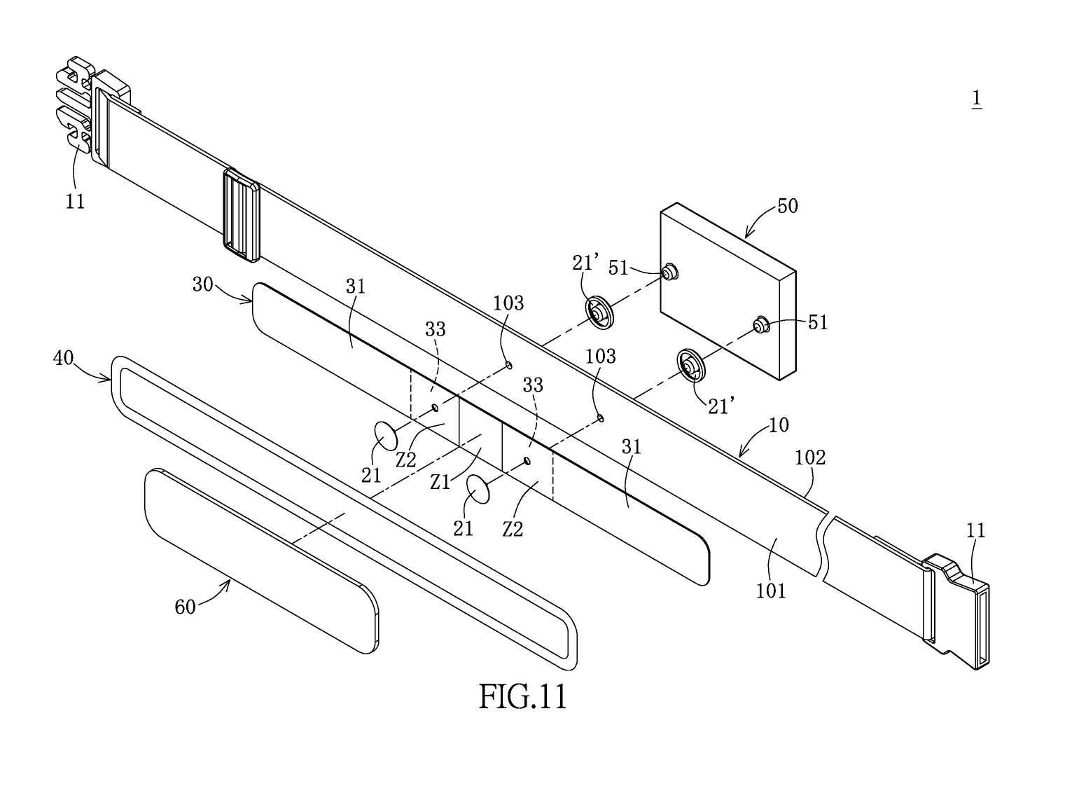

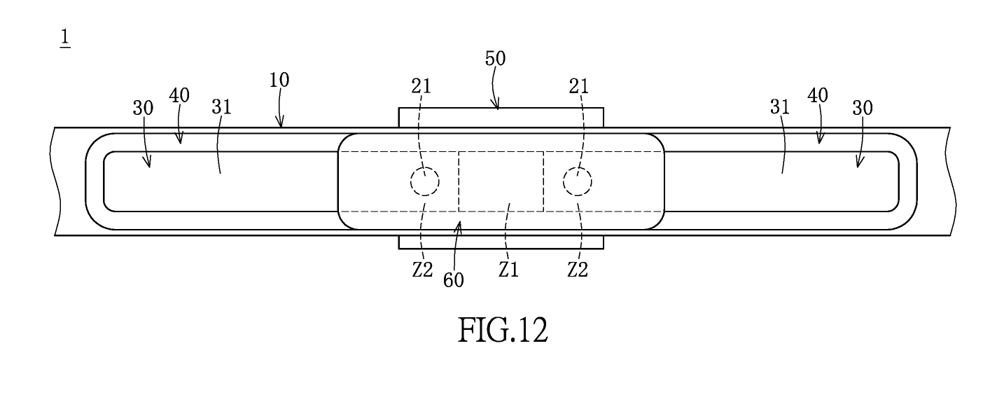

Please refer to FIGS. 11 to 12, which are schematic diagrams of a wearable device for detecting physiological signals according to still another preferred embodiment of the present invention. The wearable device 1 includes a tape-like fabric body 10, two electrically conductive units (unnumbered, refer to FIG. 2), two electric signal detection units 30, two anti-slip structures 40, and a data transmission unit 50. The tape-like fabric body 10 has two opposite surfaces defined as a first surface 101 and a second surface 102. The electric signal detection units 30 are arranged on the first surface 101, and the data transmission unit 50 is arranged on the second surface 102. The electrically conductive units 20 are arranged to pass through the tape-like fabric body 10 such that the electric signal detection units 30 can be electrically connected to the data transmission unit 50. Thus, the micro electric signals detected by the electric signal detection units 30 can be transmitted to the data transmission unit 50 by the electrically conductive units 20. The features of the electrically conductive units 20 and the data transmission unit 50 have the same features as those in the aforementioned embodiment, so that it will not be described here any further. The following will describe the details of the electric signal detection units 30.

Referring to FIG. 11, the electric signal detection units 30 has a non-conductive area Z1 at its central position and two conductive areas Z2 divided by the non-conductive area Z1. Each conductive area Z2, which is the same as the aforementioned embodiment, defines a sensing portion 31 and a connection portion 33 connected to the sensing portion 31. The sensing portion 31 is configured to come into contact with a body surface of a living body, and the connection portion 33 is electrically connected to the corresponding conductive member 21 on the first surface 101.

Referring to FIGS. 11 to 12, the countour of each anti-slip structure 40 is designed in relation to the corresponding electric signal detection unit 30. Each anti-slip structure 40 is arranged on the first surface 101 of the tape-like fabric body 10 and attached adhesively to the outer periphery of the corresponding electric signal detection unit 30. The insulating member 60 is configured to cover the non-conductive area Z1, the two connection portions 33 of the two conductive areas Z2, and the conductive members 21 on the first surface 101.

The descriptions illustrated supra set forth simply the preferred embodiments of the instant disclosure; however, the characteristics of the instant disclosure are by no means restricted thereto. All changes, alternations, or modifications conveniently considered by those skilled in the art are deemed to be encompassed within the scope of the instant disclosure delineated by the following claims.

What is claimed is:

1. A wearable device for wearing on a living body, comprising:
a tape-like fabric body having a first surface and a second surface opposite to the first surface, wherein one end thereof is configured to selectively connect the other end thereof;
at least one electrically conductive unit arranged to pass through the tape-like fabric body;
at least one electric signal detection unit arranged on the first surface, including a sensing portion and a connection portion electrically connected to the sensing portion and the electrically conductive unit, wherein the sensing portion is configured to detect micro electric signals of the living body by using an external surface thereof away from the first surface to come in contact with a body surface of the living body, and the connection portion is configured to transmit the micro electric signals to the electrically conductive unit;
an anti-slip structure arranged on the first surface of the tape- like fabric body and attached adhesively to the outer periphery of the sensing portion of the electric signal detection unit, and the anti-slip structure has a microstructure on an external surface thereof away from the first surface, wherein the microstructure is configured to come in contact with the body surface together with a sensing surface of the sensing portion; and
a data transmission unit arranged on the second surface and electrically connected to the electrically conductive unit, configured to receive the micro electric signals by the electric signal detection unit and convert the micro electric signals into the output digital signals.

2. The wearable device according to claim 1, wherein the electrically conductive unit includes two conductive members arranged on the first and second surfaces of the tape-like fabric body respectively, and the conductive member on the second surface and a conductive structure of the data transmission unit are fasteners configured to be fastened to each other.

3. The wearable device according to claim 1, wherein the electric signal detection unit is a multi-layered composite material which includes an electrically conductive fabric configured to come in contact with the body surface of the living body.

4. The wearable device according to claim 3, wherein the electric signal detection unit includes a conductive layer, an interface layer, and a base layer, the conductive layer and the base layer are arranged on two opposite surfaces of the interface layer respectively, the conductive layer is formed with a plurality of micro-holes and adapted to sense and transmit the micro electric signals, and one side of the base layer away from the interface layer is connected to the first surface of the tape-like fabric body.

5. The wearable device according to claim 2, wherein the electric signal detection unit further includes an extension portion connected between the sensing portion and the connection portion, the width of the extension portion is smaller than that of the sensing portion, and width of the sensing portion is smaller than that of the tape-like fabric body.

6. The wearable device according to claim 5, further comprising an insulating member arranged on the first surface of the tape-like fabric body to cover the conductive member on the first surface, the connection portion, the extension portion, and a part of the sensing portion of the electric signal detection unit.

7. The wearable device according to claim 1, wherein the electric signal detection unit is a tape-like structure, having a non-conductive area and two conductive areas divided by the non-conductive area, each of the conductive areas defines a sensing portion, and the two sensing portions are electrically connected to the two electrically conductive units respectively.

8. The wearable device according to claim 7, wherein each of the conductive areas further defines a connection portion which extends from the sensing portion, and the two connection portions are electrically connected to the two electrically conductive units respectively.

9. The wearable device according to claim 8, further comprising an insulating member arranged on the first surface of the tape-like fabric body to cover the non-conductive area and the two connection portions of the two conductive areas.
